(19) United States
(12) Patent Application Publication
(10) Pub. No.: US 2010/0194960 A1
(43) Pub. Date: Aug. 5, 2010

(54) CHARGE INCREASER

(75) Inventors: Hayato Nakashima, Anpachi-gun (JP); Mamoru Arimoto, Ogaki-shi (JP); Ryu Shimizu, Mizuho-shi (JP); Kaori Misawa, Kaizu-shi (JP)

Correspondence Address: DITTHAVONG MORI & STEINER, P.C., 918 Prince Street, Alexandria, VA, 22314, US

(73) Assignee: Sanyo Electric Co., Ltd., Moriguch-shi (JP)

(21) Appl. No.: 12/697,441

(22) Filed: Feb. 1, 2010

(30) Foreign Application Priority Data
Feb. 5, 2009 (JP) 2009-25160
Jan. 8, 2010 (JP) 2010-2808

(51) Int. Cl. H04N 5/335

(52) U.S. Cl. 348/311; 348/E05.091

(57) ABSTRACT

This charge increaser includes a charge supplying portion having a signal source formed by a measurement object other than visible light and supplying signal charges corresponding to the signal source and a charge increasing portion for increasing the amount of charges corresponding to the signal charges stored in the charge supplying portion by measuring the measurement object other than visible light.

### CROSS-REFERENCE TO RELATED APPLICATIONS

The priority application numbers JP2009-25160, Charge Increaser, Feb. 5, 2009, Mamoru Arimoto, Ryu Shimizu and JP2010-2808, Charge Increaser, Jan. 8, 2010, Hayato Nakashima, Mamoru Arimoto, Ryu Shimizu, Kaori Misawa, upon which this patent application is based are hereby incorporated by reference.

### BACKGROUND OF THE INVENTION

1. Field of the Invention

The present invention relates to a charge increaser, and more particularly, it relates to a charge increaser including a charge increasing portion for increasing the amount of signal charges.

2. Description of the Background Art

A charge increaser including a charge increasing portion for increasing the amount of signal charges is known in general.

An image sensor (charge increaser) including a photodiode portion having a photoelectric conversion function and a charge increasing portion (multiplying portion) for increasing the amount of charges formed by the photodiode portion receiving light (particularly visible light) by impact ionization is disclosed in general. In the conventional image sensor, the charge increasing portion increases the amount of the charges formed by the photodiode portion, thereby improving sensitivity of the image sensor.

### SUMMARY OF THE INVENTION

A charge increaser according to an aspect of the present invention includes a charge supplying portion having a signal source formed by a measurement object other than visible light and supplying signal charges corresponding to the signal source and a charge increasing portion for increasing the amount of charges corresponding to the signal charges stored in the charge supplying portion by measuring the measurement object other than visible light.

According to the aforementioned structure, sensitivity can be improved also in the apparatus measuring the measurement object other than visible light. The visible light denotes light sensible by human eyes (visual sensation). The visible light has a wavelength of at least about 360 nm and not more than about 830 nm.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a plan view of a thermosensor according to a first embodiment of the present invention;

FIG. 2 is a sectional view of the thermosensor according to the first embodiment of the present invention;

FIG. 3 is a circuit diagram of the thermosensor according to the first embodiment of the present invention;

FIG. 4 is a sectional view for illustrating an operation of the thermosensor according to the first embodiment of the present invention;

FIG. 5 is a diagram for illustrating a transfer operation of the thermosensor according to the first embodiment of the present invention;

FIG. 6 is a diagram for illustrating a multiplying operation of the thermosensor according to the first embodiment of the present invention;

FIG. 7 is a sectional view of a thermosensor according to a second embodiment of the present invention;

FIG. 8 is a timing chart for illustrating an operation of the thermosensor according to the second embodiment of the present invention;

FIG. 9 is a sectional view of a thermosensor according to a third embodiment of the present invention;

FIG. 10 is a sectional view of a thermosensor according to a fourth embodiment of the present invention;

FIG. 11 is a sectional view of a thermosensor according to a fifth embodiment of the present invention;

FIG. 12 is a sectional view of a thermosensor according to a sixth embodiment of the present invention;

FIG. 13 is a sectional view of a thermosensor according to a seventh embodiment of the present invention;

FIG. 14 is a sectional view of a thermosensor according to an eighth embodiment of the present invention;

FIG. 15 is a timing chart for illustrating an operation of the thermosensor according to the eighth embodiment of the present invention;

FIG. 16 is a sectional view of a glucose sensor according to a ninth embodiment of the present invention; and

FIG. 17 is a sectional view of a semiconductor gas sensor according to a tenth embodiment of the present invention.

### DESCRIPTION OF THE PREFERRED EMBODIMENTS

Embodiments of the present invention are now described with reference to the drawings.

### First Embodiment

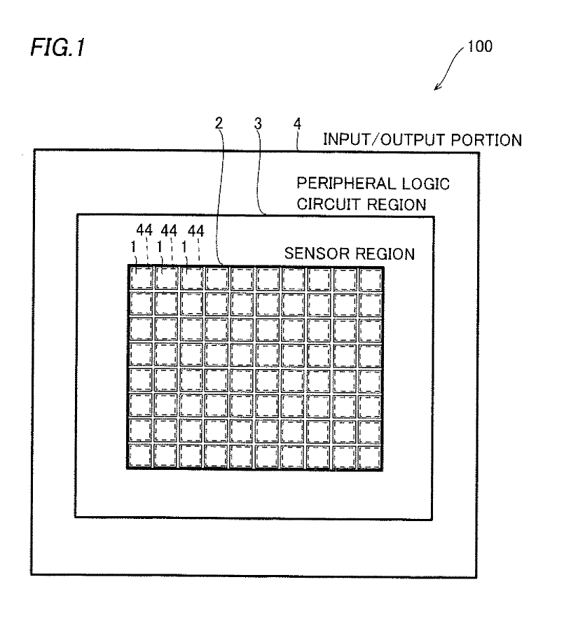

The structure of a thermosensor 100 according to a first embodiment of the present invention is now described with reference to FIGS. 1 to 3. In the first embodiment, the charge increaser according to the present invention is applied to the thermosensor 100.

As shown in FIG. 1, the thermosensor 100 includes a sensor region 2 including a plurality of thermosensor portions 1 arranged in the form of a matrix (in rows and columns), a peripheral logic circuit region 3 formed on the periphery of the sensor region 2 and an input/output portion 4.

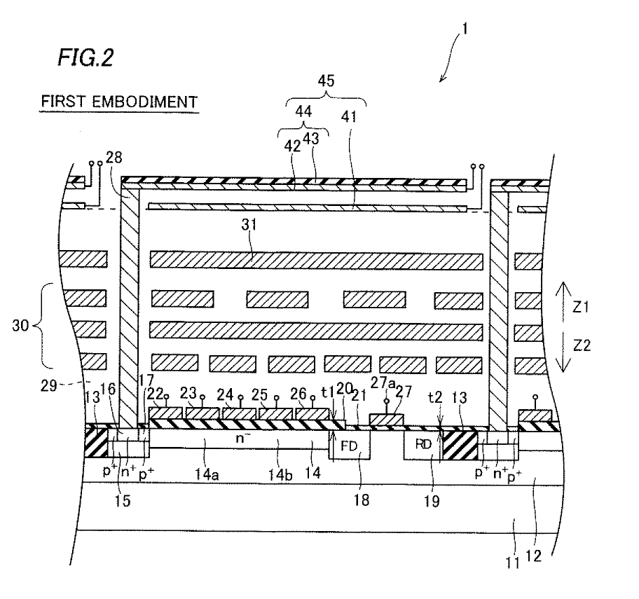

In each thermosensor portion 1, an element isolation region 13 for isolating the corresponding thermosensor portion 1 is formed on the surface of a p-type well region 12 formed on the surface of an n-type silicon substrate 11, as shown in FIG. 2. A transfer channel 14 consisting of an n⁻-type impurity region is formed on the surface of the p-type well region 12. An n-type well region 15 is formed to be adjacent to a first side of the transfer channel 14. A diffusion layer 16 consisting of an n⁺-type impurity region and a p⁺ layer 17 consisting of a p⁺-type impurity region are formed on the surface of the n-type well region 15. The p⁺ layer 17 is formed to cover the periphery of the diffusion layer 16. The p⁺ layer 17 has a function of capturing a dark current generated from an interface state around the surface of the n-type well region 15. Thus, supply of noise resulting from the dark current to the transfer channel 14 is suppressed. The n-type silicon substrate 11 is an example of the “semiconductor substrate” in the present invention. The transfer channel 14 is an example of the “charge transfer region” in the present invention.

A floating diffusion region (FD region) 18 consisting of an n-type impurity region is formed to be adjacent to a second side of the transfer channel 14. A reset drain region (RD region) 19 is formed at a prescribed interval from the FD region 18.

An insulating film 20 consisting of a thermal silicon oxide film (SiO₂ film) prepared by thermally oxidizing the surface of the silicon (Si) substrate 11 is formed on a portion of the surface of the p-type well region 12 corresponding to a region from the surface of the transfer channel 14 to the surface of the FD region 18. The insulating film 20 has a thickness t1 of about 60 nm. Another insulating film 21 having a thickness t2 of not more than about 7 nm, smaller than the thickness t1 of the insulating film 20, is formed on another portion of the surface of the p-type well region 12 corresponding to a region from the surface of the FD region 18 to the surface of the RD region 19.

A transfer gate electrode 22, a multiplier gate electrode 23, another transfer gate electrode 24, a storage gate electrode 25 and a read gate electrode 26 are formed on the surface of the insulating film 20 in this order from the side of the n-type well region 15 toward the side of the FD region 18. An electron multiplying portion 14a is provided in a portion of the transfer channel 14 located under the multiplier gate electrode 23, while an electron storage portion 14b is provided in another portion of the transfer channel 14 located under the storage gate electrode 25. The multiplier gate electrode 23 is an example of the “increasing electrode” in the present invention. The electron multiplying portion 14a is an example of the “charge increasing portion” in the present invention.

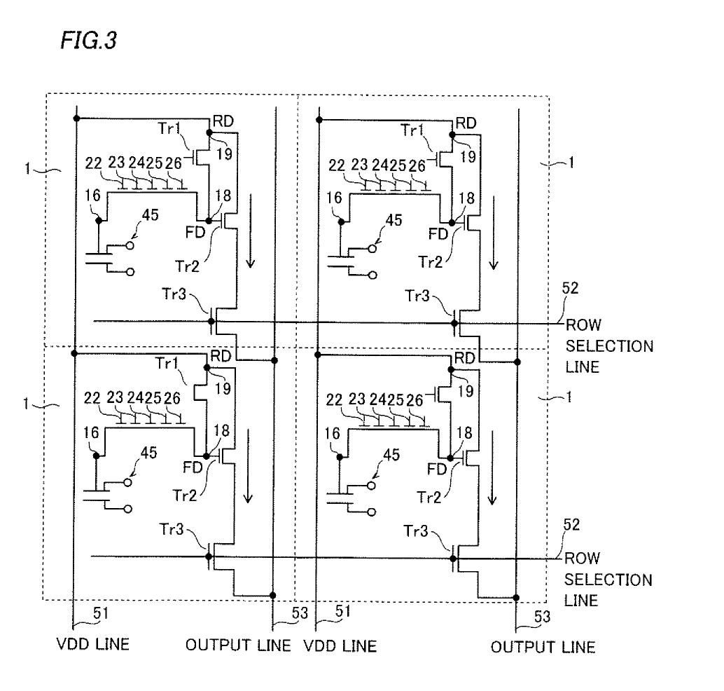

A reset gate electrode 27 is formed on a portion of the surface of the insulating film 21 corresponding to the space between the FD region 18 and the RD region 19. The FD region 18, the RD region 19 and the reset gate electrode 27 constitute a reset transistor Tr1 (see FIG. 3).

A connecting wire 28 consisting of a metal layer to be connected to an upper electrode 42 described later is formed on the surface of the n-type well region 15 (diffusion layer 16). A trilaminar wiring layer 30 is formed on the surfaces of the transfer gate electrode 22, the multiplier gate electrode 23, the transfer gate electrode 24, the storage gate electrode 25, the read gate electrode 26 and the reset gate electrode 27 through an insulating film 29. A shielding layer 31 consisting of a metal layer is formed on the surface of the wiring layer 30 through the insulating film 29, to cover the transfer gate electrode 22, the multiplier gate electrode 23, the transfer gate electrode 24, the storage gate electrode 25, the read gate electrode 26 and the reset gate electrode 27. The shielding layer 31 has a function of suppressing incidence of light (particularly visible light) upon the electron multiplying portion 14a.

A lower electrode 41 is formed on the surface of the shielding layer 31 to be opposed to the upper electrode 42 described later through the insulating film 29. The lower electrode 41 is made of a metal such as nickel (Ni). The lower electrode 41 is so formed that a voltage is applied thereto. The upper electrode 42 is provided to be connected to the connecting wire 28 and to be opposed to the lower electrode 41 at a prescribed interval. The upper electrode 42 is so formed that a voltage is applied thereto. No component is provided in the space between the lower electrode 41 and the upper electrode 42. In other words, the upper electrode 42 is formed on a portion upwardly separated (along arrow Z1) from the n-type silicon substrate 11 provided with the lower electrode 41 etc. The lower electrode 41 and the upper electrode 42 form a capacitance. The lower electrode 41 is an example of the “second electrode” in the present invention.

An insulating film 43 made of a material having a thermal expansion coefficient different from that of the upper electrode 42 is formed on the surface of the upper electrode 42. The insulating film 43 consists of a silicon nitride (SiN) film, for example. The upper electrode 42 and the insulating film 43 have cantilever structures, to constitute a cantilever electrode 44. The cantilever electrode 44 and the lower electrode 41 constitute a charge supplying portion 45. The cantilever electrode 44 (upper electrode 42) is formed to extend from the diffusion layer 16 up to the element isolation region 13 toward the side of the FD region 18 in plan view. In other words, the upper electrode 42 is provided to overlap with the electron multiplying portion 14a. As shown in FIG. 1, the cantilever electrode 44 is substantially rectangular in plan view, and formed to cover substantially the overall region of the corresponding thermosensor portion 1. The cantilever electrode 44 is an example of the “first electrode” in the present invention.

According to the first embodiment, the cantilever electrode 44 is formed to be bent and deformed due to the difference between the thermal expansion coefficients of the upper electrode 42 and the insulating film 43 by detecting heat (infrared radiation). The infrared radiation has a wavelength longer than the wavelength (at least about 620 nm and not more than about 750 nm) of red visible light. The capacitance between the upper electrode 42 and the lower electrode 41 changes due to the deformation of the cantilever electrode 44, so that electrons stored in the upper electrode 42 are supplied to the transfer channel 14 through the connecting wire 28 and the diffusion layer 16. The electrons supplied to the transfer channel 14 are multiplied (increased) in the electron multiplying portion 14a.

As shown in FIG. 3, each thermosensor portion 1 includes the transfer gate electrode 22, the multiplier gate electrode 23, the transfer gate electrode 24, the storage gate electrode 25, the read gate electrode 26, the reset transistor Tr1, an amplifying transistor Tr2 and a selection transistor Tr3.

A reset gate line 27a (see FIG. 2) is connected to the reset gate electrode 27 of the reset transistor Tr1, to supply a reset signal. The RD region 19 functions as the drain of the reset transistor Tr1, and is connected to a power supply (VDD) line 51. The FD region 18 functions as the source of the reset transistor Tr1 and the drain of the read gate electrode 26, and is connected with the gate of the amplifying transistor Tr2. The source of the selection transistor Tr3 is connected to the drain of the amplifying transistor Tr2. A row selection line 52 and an output line 53 are connected to the gate and the drain of the selection transistor Tr3 respectively.

The thermosensor 100 is so formed that the amplifying transistor Tr2 amplifies a signal in each thermosensor portion 1 due to the circuit structure shown in FIG. 3. The read gate electrode 26 is on-off-controlled every row, while the remaining gate electrodes 22 to 25 other than the read gate electrode 26 are simultaneously on-off-controlled with respect to the overall thermosensor portion 1.

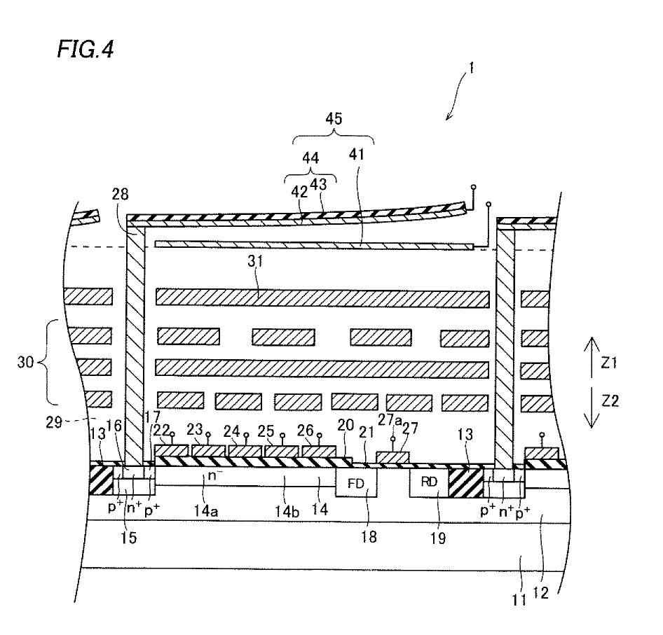

The operation of each thermosensor portion 1 detecting heat (infrared radiation) is now described with reference to FIGS. 2 and 4.

First, a prescribed potential difference is caused between the upper electrode 42 and the lower electrode 41, as shown in FIG. 2. For example, a potential difference of about 3 V is caused between the upper electrode 42 and the lower electrode 41. Consequently, electrons corresponding to the potential difference of about 3 V are stored in the upper electrode 42 and the lower electrode 41 respectively.

Then, the cantilever electrode 44 detects heat (infrared radiation). Thus, when the temperature of the cantilever electrode 44 is increased, the cantilever electrode 44 is gradually bent upward (along arrow Z1) due to the difference between the thermal expansion coefficients of the upper electrode 42 and the insulating film 43 of the cantilever electrode 44. Consequently, the capacitance between the upper electrode 42 of the cantilever electrode 44 and the lower electrode 41 is reduced. Thus, the amount of the electrons stored in the upper electrode 42 is reduced, and surplus electrons are supplied from the upper electrode 42 to the transfer channel 14 through the connecting wire 28, the diffusion layer 16 and the n-type well region 15 as a current.

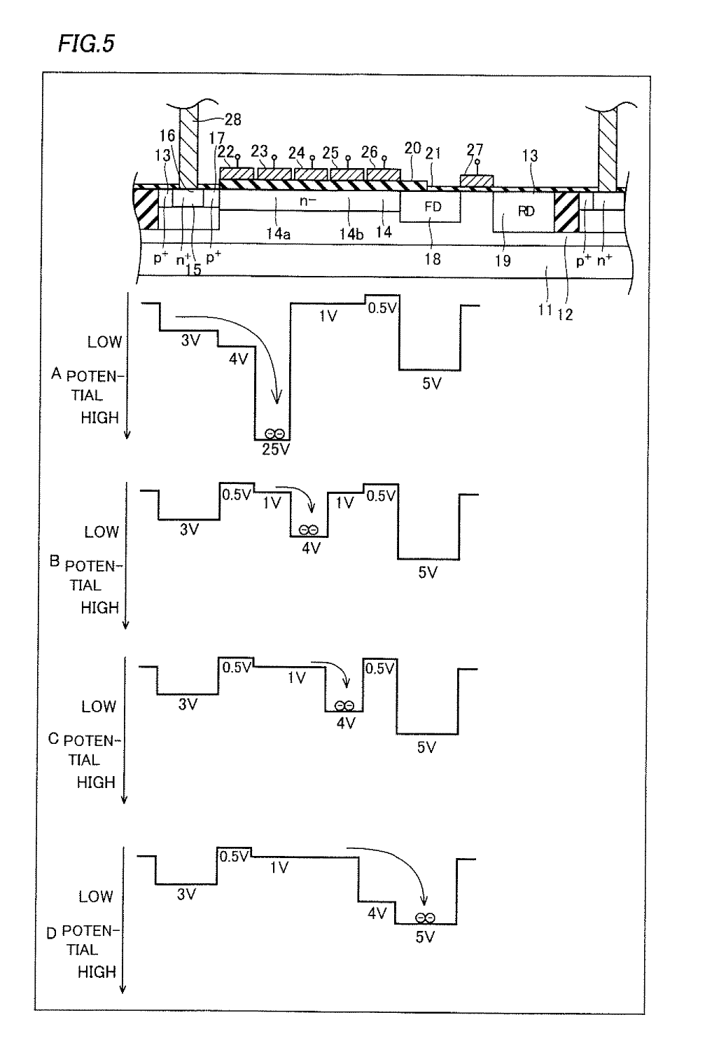

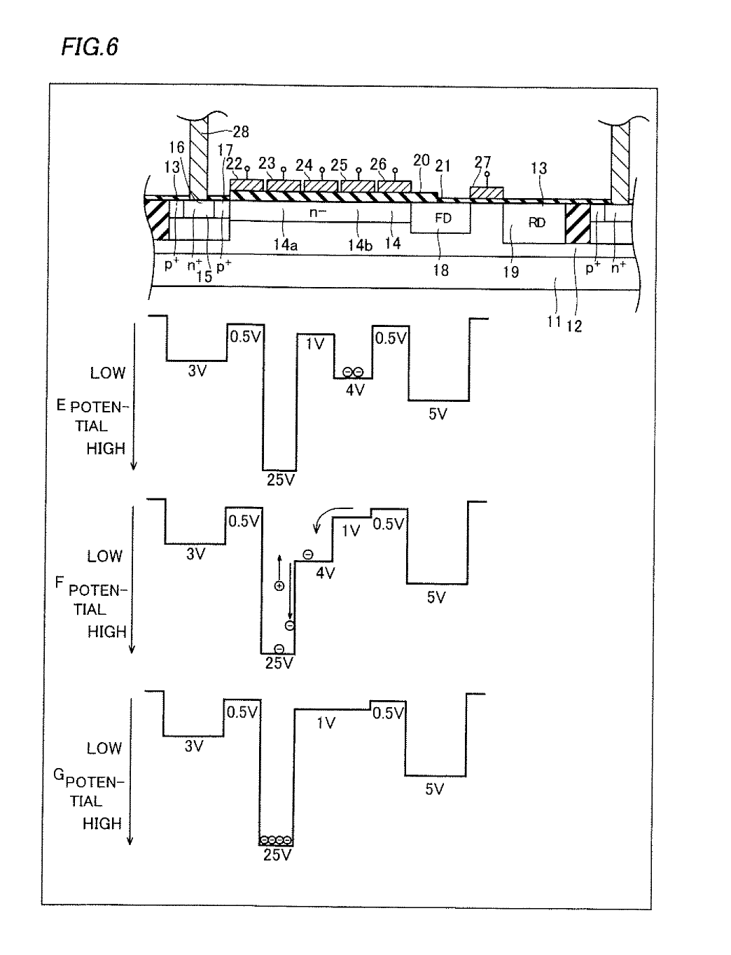

Transfer and multiplying operations for the electrons supplied to the transfer channel 14 are now described with reference to FIGS. 5 and 6.

First, the electron transfer operation is described. In a period A shown in FIG. 5, the electrons supplied from the cantilever electrode 44 are transferred to the portion (electron multiplying portion 14a) of the transfer channel 14 located under the multiplier gate electrode 23 having a high potential. Then, the electrons are transferred to the portion of the transfer channel 14 located under the transfer gate electrode 24 in a period B, and transferred to the portion (electron storage portion 14b) of the transfer channel 14 located under the multiplier gate electrode 25 in a period C. Thereafter the electrons are transferred to the FD region 18 through the read gate electrode 26 in a period D.

The electron multiplying operation is now described. The electron multiplying operation is performed in the portion of the transfer channel 14 located between the multiplier gate electrode 23 and the storage gate electrode 25. More specifically, the operation is performed in periods E, F and G shown in FIG. 6 from the state of the period C when the electrons have been held in the electron storage portion 14b located under the storage gate electrode 25. In other words, the potential of the electron multiplying portion 14a located under the multiplier gate electrode 23 is adjusted to about 25 V in the period E, while the potential of the portion of the transfer channel 14 located under the transfer gate electrode 24 is adjusted to about 4 V in the period F. Thereafter the potential of the electron storage portion 14b located under the storage gate electrode 25 is adjusted to about 1 V, whereby the electrons stored in the electron storage portion 14b are transferred to the electron multiplying portion 14a (about 25 V) located under the multiplier gate electrode 23 through the portion (about 4 V) of the transfer channel 14 located under the transfer gate electrode 24. At this time, the electrons are multiplied by impact ionization.

The transfer gate electrode 24 is brought into an OFF-state in the period G, thereby completing the multiplying operation. The aforementioned electron transfer operation is so performed from this state that the multiplied electrons are transferred to the FD region 18. In the electron multiplying operation, the potentials of the portions of the transfer channel 14 located under the transfer gate electrode 22 and the read gate electrode 26 respectively are adjusted to about 0.5 V, whereby the electrons can be inhibited from moving toward the n-type well region 15 as well as toward the FD region 18.

Signal charges formed by the electrons multiplied in the aforementioned manner are read as a voltage signal through the FD region 18 due to the aforementioned read operation. The electron transfer operation between the electron multiplying portion 14a and the electron storage portion 14b is performed a plurality of times (about 400 times, for example), whereby the electrons supplied from the cantilever electrode 44 are multiplied to about 2000 times. As shown in FIG. 1, the thermosensor portions 1 are so arranged in the form of a matrix that the thermosensor 100 can measure planar distribution of heat.

The thermosensor 100 according to the first embodiment can attain the following effects:

(1) The thermosensor 100 includes the charge supplying portion 45 (upper electrode 42) supplying electrons corresponding to heat (infrared radiation) and the electron multiplying portion 14a for increasing the amount of the electrons stored in the charge supplying portion 45 by measuring the heat (infrared radiation). Thus, even if the measurement object is at a low temperature, the electron multiplying portion 14a can multiply a small amount of electrons corresponding to the low temperature, whereby the thermosensor 100 can correctly detect (measure) the low temperature. Consequently, sensitivity of the thermosensor 100 detecting the heat (infrared radiation) can be improved. Even if the temperature of the measurement object slightly changes, the electron multiplying portion 14a can multiply a small amount of electrons corresponding to the slight change of the temperature, whereby the thermosensor 100 can detect the small change of the temperature.

(2) The charge supplying portion 45 is arranged on the portion upwardly separated from the n-type silicon substrate 11. For example, a photoelectric conversion portion such as a photodiode can be formed on the same substrate as the electron multiplying portion 14a and the electron storage portion 14b, while it may be difficult to form a structure converting heat (infrared radiation, for example) or the like other than light (visible light) to electrons on the same substrate as the electron multiplying portion 14a and the electron storage portion 14b. Therefore, the charge supplying portion 45 is so arranged on the portion upwardly separated from the n-type silicon substrate 11 that the same can be easily arranged on the thermosensor 100. Further, a highly sensitive sensor capable of detecting a change in ultraviolet radiation or another environmental factor other than infrared radiation can be easily implemented by replacing the charge supplying portion 45 with a sensor detecting a desired measurement object.

(3) The charge supplying portion 45 is provided above the n-type silicon substrate 11, so that the electrons stored in the charge supplying portion 45 are transferred to the electron multiplying portion 14a provided on the upper surface of the n-type silicon substrate 11 and the amount thereof is increased. Thus, the charge supplying portion 45 and the electron multiplying portion 14a are provided only on a first side of the n-type silicon substrate 11, whereby the structure of the thermosensor 100 can be simplified dissimilarly to a case where the charge supplying portion 45 and the electron multiplying portion 14a are separately provided on the first side and a second side of the n-type silicon substrate 11.

(4) The thermosensor 100 includes the connecting wire 28 connecting the charge supplying portion 45 and the n-type silicon substrate 11 with each other. Thus, the electrons having been stored in the charge supplying portion 45 can be easily supplied to the transfer channel 14 provided on the n-type silicon substrate 11 through the connecting wire 28.

(5) The charge supplying portion 45 and the electron multiplying portion 14a are arranged to overlap with each other in plan view. Thus, the charge supplying portion 45 and the electron multiplying portion 14a so overlap with each other that the charge supplying portion 45 can suppress incidence of light (particularly visible light) resulting in noise upon the electron multiplying portion 14a.

(6) The thermosensor 100 includes the shielding layer 31 provided between the charge supplying portion 45 and the electron multiplying portion 14a for suppressing incidence of light upon the electron multiplying portion 14a. Thus, the shielding layer 31 can reliably suppress incidence of light (particularly visible light) resulting in noise upon the electron multiplying portion 14a.

(7) The electron multiplying portion 14a is provided on the portion of the transfer channel 14 located under the multiplier gate electrode 23, for multiplying the electrons stored in the charge supplying portion 45 by voltage application to the multiplier gate electrode 23. Thus, the electrons can be easily multiplied by impact ionization in the portion of the transfer channel 14 located under the multiplier gate electrode 23 by voltage application to the multiplier gate electrode 23.

(8) The charge supplying portion 45 is constituted of the cantilever electrode 44 consisting of the upper electrode 42 and the insulating film 43 having different thermal expansion coefficients and the lower electrode 41 opposed to the cantilever electrode 44, so that the electron multiplying portion 14a increases the amount of the electrons resulting from the change in the capacitance between the upper electrode 42 and the lower electrode 41 upon deformation of the cantilever electrode 44 due to heat (infrared radiation). Thus, a change in the heat (infrared radiation) is reflected on the change in the capacitance between the upper electrode 42 and the lower electrode 41, whereby the change in the heat (infrared radiation) can be easily extracted as electrons.

### Second Embodiment

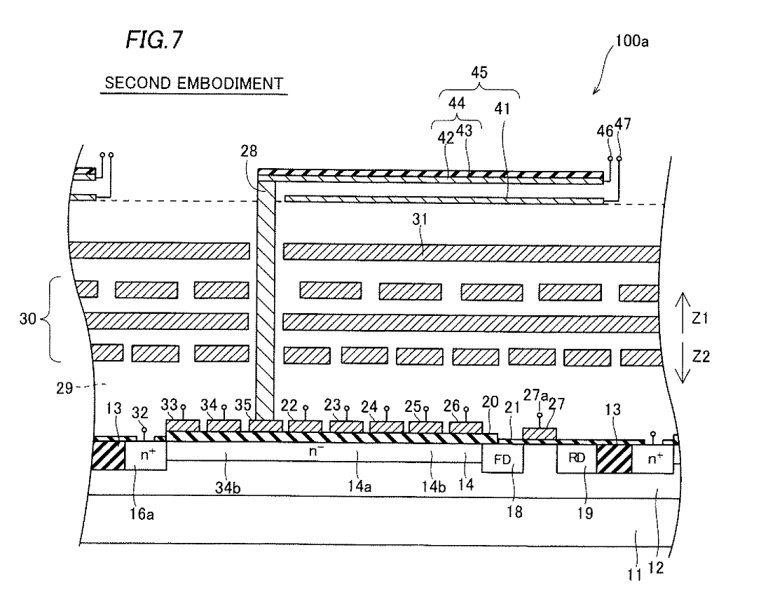

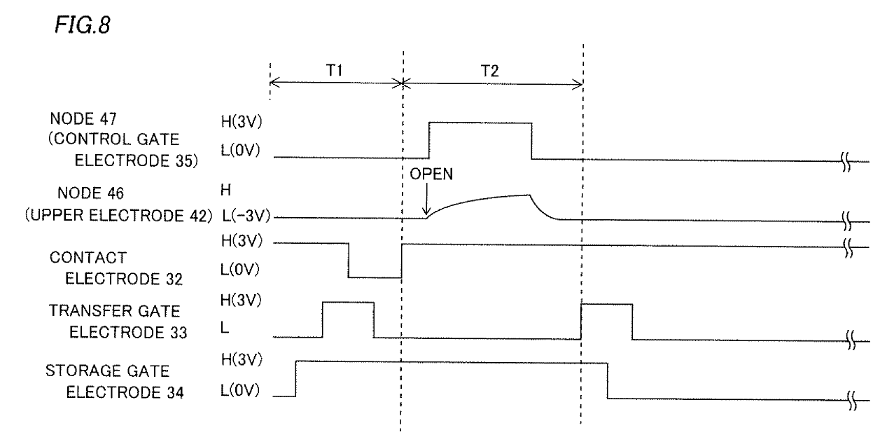

A thermosensor 100a according to a second embodiment of the present invention is now described with reference to FIGS. 7 and 8. According to the second embodiment, the potential of an upper electrode 42 corresponding to a potential difference caused between electrodes of a charge supplying portion 45 is applied to a control gate electrode 35, dissimilarly to the aforementioned first embodiment supplying the electrons stored in the charge supplying portion 45 to the transfer channel 14.

In the thermosensor 100a according to the second embodiment of the present invention, a diffusion layer 16a consisting of an n⁺-type impurity region is formed on the surface of a p-type well region 12 to be adjacent to a transfer channel 14, as shown in FIG. 7. A contact electrode 32 is provided on the diffusion layer 16a. A transfer gate electrode 33, a storage gate electrode 34 and the control gate electrode 35 are provided on the surface of a portion of an insulating film 20 located between a transfer gate electrode 22 and the diffusion layer 16a. A connecting wire 28 is connected to the control gate electrode 35. The capacitance between the upper electrode 42 and a lower electrode 41 is changed due to deformation of a cantilever electrode 44, so that the potential formed in the upper electrode 42 is applied to the control gate electrode 35. The remaining structure of the second embodiment is similar to that of the aforementioned first embodiment.

An operation of the thermosensor 100a detecting heat (infrared radiation) is now described with reference to FIG. 8. Nodes 46 and 47 are connected to a control circuit (not shown) for an applied voltage.

(Period T1)

First, a voltage of about −3 V is applied to the node 46 connected to the upper electrode 42. Thus, a portion of the transfer channel 14 located under the control gate electrode 35 is brought into an OFF-state. The node 47 connected to the lower electrode 41 is grounded (0 V).

Then, a voltage of about 3 V is successively applied to the contact electrode 32, the storage gate electrode 34 and the transfer gate electrode 33, and the potential of the contact electrode 32 is thereafter set to 0 V. Thus, electrons introduced into the transfer channel 14 from the contact electrode 32 through the diffusion layer 16a are stored in an electron storage portion 34b formed under the storage gate electrode 34. Then, the potential of the transfer gate electrode 33 is set to 0 V, and the potential of the contact electrode 32 is thereafter set to about 3 V again.

(Period T2)

The node 46 connected to the upper electrode 42 is brought into an open state, while a voltage of about 3 V is applied to the node 47 connected to the lower electrode 41.

When the cantilever electrode 44 (upper electrode 42) receives infrared radiation and accumulates heat, the upper electrode 42 is gradually bent upward (along arrow Z1 in FIG. 7), whereby the potential thereof is increased from about −3 V. Consequently, the portion of the transfer channel 14 located under the control gate electrode 35 is brought into an ON-state, and the electrons having been stored in the electron storage portion 34b are transferred to an electron multiplying portion 14a. The amount of the electrons transferred to the electron multiplying portion 14a corresponds to the potential of the upper electrode 42 increased from about −3 V. In other words, the electrons having been stored in the electron storage portion 34b are transferred to the electron multiplying portion 14a by the infrared radiation received by the upper electrode 42. Thus, the thermosensor 100a can measure heat.

A transfer operation between the electron multiplying portion 14a and an electron storage portion 14b is performed a plurality of times (about 400 times, for example) similarly to the operations shown in FIGS. 5 and 6, so that the electrons transferred to the electron multiplying portion 14a are multiplied to about 2000 times. Thereafter the multiplied electrons are read as a voltage signal by the aforementioned read operation through the FD region 18.

The thermosensor 100a according to the second embodiment can attain the following effect:

(9) The thermosensor 100a so measures the heat (infrared radiation) that the potential of the charge supplying portion 45 (upper electrode 42) is applied to the control gate electrode 35 and electrons corresponding to the potential of the upper electrode 42 are supplied from the diffusion layer 16a to the transfer channel 14 and multiplied (increased in amount) in the electron multiplying portion 14a. Thus, even if the measurement object is at a low temperature, the electron multiplying portion 14a can multiply electrons, corresponding to the low temperature, supplied from the diffusion layer 16a, whereby the thermosensor 100a can correctly measure the low temperature. Consequently, sensitivity of the thermosensor 100a detecting the heat (infrared radiation) can be improved.

### Third Embodiment

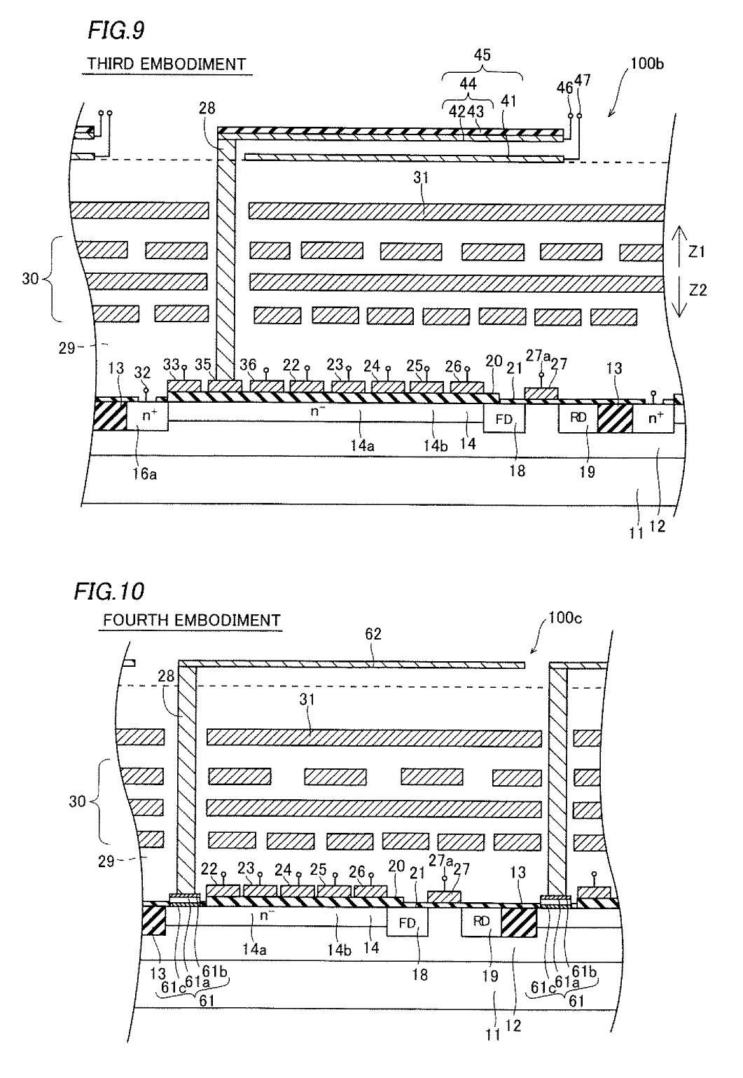

A thermosensor 100b according to a third embodiment of the present invention is now described with reference to FIG. 9. According to the third embodiment, electrons stored in a diffusion layer 16a are supplied to an electron multiplying portion 14a, dissimilarly to the aforementioned second embodiment supplying the electrons stored in the electron storage portion 34b to the electron multiplying portion 14a.

In the thermosensor 100b according to the third embodiment of the present invention, a transfer gate electrode 33, a control gate electrode 35 and another transfer gate electrode 36 are successively provided on the surface of a portion of an insulating film 20 located between a transfer gate electrode 22 and the diffusion layer 16a. A connecting wire 28 is connected to the control gate electrode 35. The capacitance between an upper electrode 42 and a lower electrode 41 is changed due to deformation of a cantilever electrode 44, so that a potential formed in the upper electrode 42 is applied to the control gate electrode 35 through the connecting wire 28 as a voltage. The remaining structure of the third embodiment is similar to that of the aforementioned second embodiment.

An operation of the thermosensor 100b according to the third embodiment of the present invention is now described. The cantilever electrode 44 (upper electrode 42) receives infrared radiation and accumulates heat so that the upper electrode 42 is deformed to be gradually bent upward (along arrow Z1 in FIG. 9), whereby the potential of the upper electrode 42 is increased from about −3 V. Consequently, a potential barrier under the control electrode 35 is reduced by the potential of the upper electrode 42 increased from about −3 V, and electrons having been stored in the diffusion layer 16a are transferred to the electron multiplying portion 14a through a portion of a transfer channel 14 located under the transfer gate electrode 33 and multiplied.

The effect of the third embodiment is similar to that of the aforementioned second embodiment.

### Fourth Embodiment

A thermosensor 100c according to a fourth embodiment of the present invention is now described with reference to FIG. 10. According to the fourth embodiment, a pyroelectric detector 61 detects heat (infrared radiation), dissimilarly to the aforementioned first to third embodiments each detecting heat (infrared radiation) by electrons resulting from the change in the capacitance between the upper electrode 42 and the lower electrode 41.

In the thermosensor 100c according to the fourth embodiment of the present invention, the pyroelectric detector 61 is directly formed on the surface of a transfer channel 14 to be adjacent to a transfer gate electrode 22, as shown in FIG. 10. The pyroelectric detector 61 is formed by a ferroelectric film 61a consisting of an SBT film (SrBi₂Ta₂O₉ film) material having hysteresis characteristics as well as a first electrode 61b and a second electrode 61c holding the ferroelectric film 61a therebetween. The first electrode 61b is made of chromium (Cr) or a nickel-chromium alloy (Ni—Cr), and the second electrode 61c is made of platinum (Pt). The pyroelectric detector 61 is connected with a connecting wire 28 provided with a heat-sensitive portion 62 made of a metal or the like. The thermosensor 100c is provided with no n-type well region 15 (see FIG. 2), no diffusion layer 16 and no p⁺ layer 17, dissimilarly to the aforementioned first embodiment. The pyroelectric detector 61 is an example of the “charge supplying portion” in the present invention. The remaining structure of the fourth embodiment is similar to that of the aforementioned first embodiment.

An operation of the thermosensor 100c according to the fourth embodiment of the present invention is now described. When the heat-sensitive portion 62 receives infrared radiation and accumulates heat, the heat is transmitted to the pyroelectric detector 61. Thus, spontaneous polarization of the ferroelectric film 61a is changed, to change the polarization state between the first electrode 61b and the second electrode 61c. Consequently, electrons, included in those having been previously stored in the surface of the pyroelectric detector 61, corresponding to the change (detected heat) in the polarization state of the ferroelectric film 61a are transferred to an electron multiplying portion 14a and multiplied.

The thermosensor 100c according to the fourth embodiment can attain the following effect:

(10) The pyroelectric detector 61 is directly provided on the surface of the transfer channel 14, so that the electrons output from the pyroelectric detector 61 detecting heat (infrared radiation) are directly supplied from the pyroelectric detector 61 to the transfer channel 14 and multiplied by the electron multiplying portion 14a. Thus, the electrons are directly supplied from the pyroelectric detector 61 to the transfer channel 14 dissimilarly to a case of providing an n-type diffusion layer, for example, and multiplying electrons supplied from a charge supplying portion through the n-type diffusion layer, whereby the structure of the thermosensor 100c can be simplified.

### Fifth Embodiment

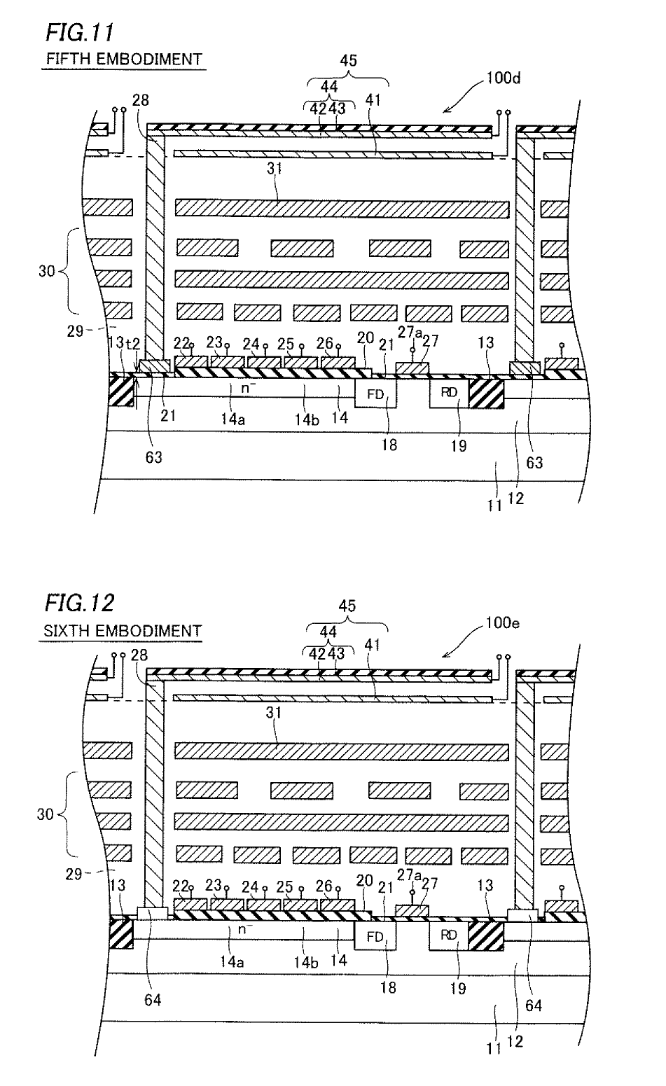

A thermosensor 100d according to a fifth embodiment of the present invention is now described with reference to FIG. 11. According to the fifth embodiment, electrons stored in an upper electrode 42 are supplied to a transfer channel 14 by tunneling, dissimilarly to the aforementioned fourth embodiment supplying the electrons from the pyroelectric detector 61 directly formed on the transfer channel 14.

In the thermosensor 100d according to the fifth embodiment of the present invention, an insulating film 21 having a thickness t2 is formed on the surface of a region of the transfer channel 14 adjacent to a transfer gate electrode 22 similarly to the surfaces of an FD region 18 and an RD region 19, as shown in FIG. 11. An electrode 63 is formed on the surface of the insulating film 21. In a portion of the insulating film 21 located under the electrode 63, the thickness t2 is so adjusted that electrons can move from the electrode 63 toward the transfer channel 14 by tunneling. The tunneling of the electrons from the electrode 63 toward the transfer channel 14 may be direct tunneling (DT), or Fowler-Nordheim (FN) tunneling easily caused due to voltage application to the electrode 63. The electrons stored in the upper electrode 42 are directly supplied to the transfer channel 14 by tunneling through a connecting wire 28 and the electrode 63, and multiplied in an electron multiplying portion 14a.

The thermosensor 100d according to the fifth embodiment can attain the following effect:

(11) The electrons stored in the upper electrode 42 are directly supplied from the connecting wire 28 to the transfer channel 14 by tunneling, and multiplied in the electron multiplying portion 14a. Thus, the electrons are directly supplied from the connecting wire 28 to the transfer channel 14 dissimilarly to a case of providing an n-type diffusion layer, for example, and multiplying electrons supplied through the n-type diffusion layer, whereby the structure of the thermosensor 100d can be simplified.

### Sixth Embodiment

A thermosensor 100e according to a sixth embodiment of the present invention is now described with reference to FIG. 12. According to the sixth embodiment, electrons stored in an upper electrode 42 are directly supplied to a transfer channel 14 through a barrier layer 64, dissimilarly to the aforementioned fourth embodiment directly supplying the electrons from the pyroelectric detector 61 to the transfer channel 14.

In the thermosensor 100e according to the sixth embodiment of the present invention, the barrier layer 64 is formed on the surface of a region of the transfer channel 14 adjacent to a transfer gate electrode 22, as shown in FIG. 12. A connecting wire 28 is provided on the surface of the barrier layer 64. The barrier layer 64 has a function of reducing resistance between the connecting wire 28 and the transfer channel 14. More specifically, the barrier layer 64 is made of tungsten (W), titanium nitride (TiN) or tantalum nitride (TaN). When made of tungsten, the barrier layer 64 consists of a single tungsten layer. When the barrier layer 64 is made of TiN having relatively high resistance, on the other hand, a TiN upper layer is formed on a titanium (Ti) lower layer. Electrons stored in the upper electrode 42 are supplied to an electron multiplying portion 14a through the connecting wire 28 and the barrier layer 64, and multiplied.

The thermosensor 100e according to the sixth embodiment can attain the following effect:

(12) The thermosensor 100e includes the barrier layer 64 provided between the connecting wire 28 and the transfer channel 14 for reducing the resistance between the connecting wire 28 and the transfer channel 14. Thus, the resistance between the connecting wire 28 and the transfer channel 14 is so reduced that a current can be easily fed from the upper electrode 42 to the transfer channel 14, whereby reliability of the thermosensor 100e can be further improved.

### Seventh Embodiment

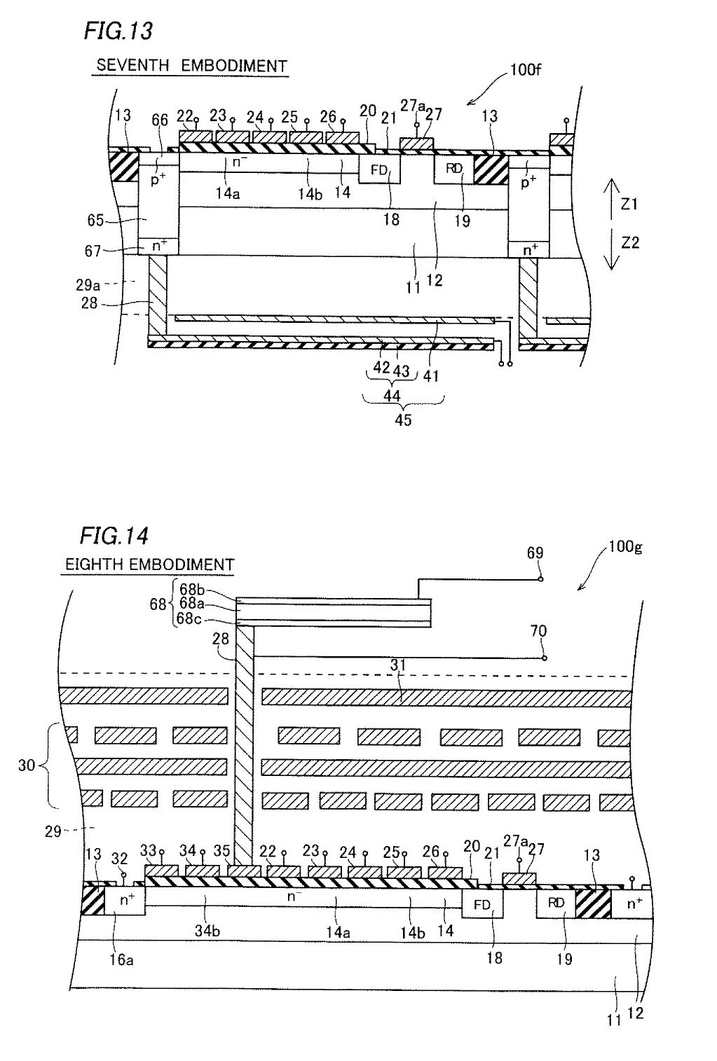

A thermosensor 100f according to a seventh embodiment of the present invention is now described with reference to FIG. 13. According to the seventh embodiment, a charge supplying portion 45 is provided on a portion downwardly separated from an n-type silicon substrate 11, dissimilarly to the aforementioned first to sixth embodiments each having the charge supplying portion 45 provided on the portion upwardly separated from the n-type silicon substrate 11.

In the thermosensor 100f according to the seventh embodiment of the present invention, an n-type well region 65 is formed in the n-type silicon substrate 11, as shown in FIG. 13. A p⁺ layer 66 is formed on the surface of the n-type well region 65 along arrow Z1, while a diffusion layer 67 consisting of an n⁺-type impurity region is formed on the surface thereof along arrow Z2. A connecting wire 28 is formed on the surface of the diffusion layer 67 along arrow Z2. An insulating film 29a is formed on the surface of the n-type silicon substrate 11 along arrow Z2, and a lower electrode 41 is formed on the surface of the insulating film 29a. A cantilever electrode 44 consisting of an upper electrode 42 and an insulating film 43 is provided to be connected to the connecting wire 28 and opposed to the lower electrode 41. The cantilever electrode 44 and the lower electrode 41 constitute the charge supplying portion 45. Electrons stored in the upper electrode 42 are supplied to an electron multiplying portion 14a through the connecting wire 28, the diffusion layer 67 and the n-type well region 65, and multiplied. The remaining structure and the remaining operation of the seventh embodiment are similar to those of the aforementioned first embodiment.

The thermosensor 100f according to the seventh embodiment can attain the following effect:

(13) The charge supplying portion 45 is so provided under the n-type silicon substrate 11 that light (particularly visible light) resulting in noise can be inhibited from entering the electron multiplying portion 14a from under the n-type silicon substrate 11. In particular, the charge supplying portion 45 and the electron multiplying portion 14a are provided to overlap with each other, whereby the aforementioned effect can be more remarkably attained.

### Eighth Embodiment

A thermosensor 100g according to an eighth embodiment of the present invention is now described with reference to FIG. 14. According to the eighth embodiment, a voltage resulting from electrons stored in a pyroelectric detector 68 is applied to a control gate electrode 35, dissimilarly to the aforementioned second embodiment applying the voltage resulting from the electrons stored in the upper electrode 42 to the control gate electrode 35.

In the thermosensor 100g according to the eighth embodiment of the present invention, a connecting wire 28 is provided on the surface of the control gate electrode 35, as shown in FIG. 14. The connecting wire 28 is provided with the pyroelectric detector 68. The pyroelectric detector 68 is formed by a ferroelectric film 68a consisting of an SBT film (SrBi₂Ta₂O₉ film) material having hysteresis characteristics as well as a first electrode 68b and a second electrode 68c holding the ferroelectric film 68a therebetween. The first electrode 68b is made of chromium (Cr) or a nickel-chromium alloy (Ni—Cr), and the second electrode 68c is made of platinum (Pt). The pyroelectric detector 68 is an example of the “charge supplying portion” in the present invention.

The first electrode 68b is connected to a node 69, which is regularly grounded. The second electrode 68c is connected to the control gate electrode 35 as well as to a node 70, which is held in either a grounded state or an open state. The remaining structure of the eighth embodiment is similar to that of the aforementioned second embodiment.

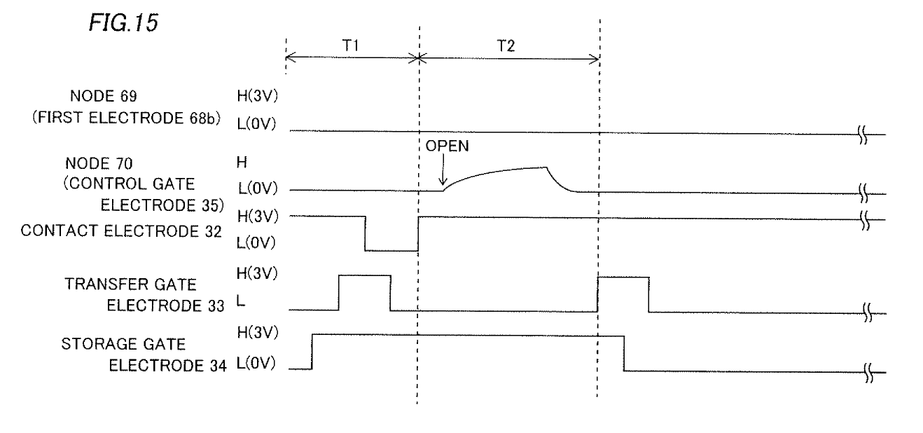

An operation of the thermosensor 100g detecting heat (infrared radiation) is now described with reference to FIG. 15. The nodes 69 and 70 are connected to a control circuit (not shown) for an applied voltage.

(Period T1)

The node 69 connected to the first electrode 68b and the node 70 connected to the second electrode 68c are maintained at grounded (0 V) states.

A voltage of about 3 V is successively applied to a contact electrode 32, a storage gate electrode 34 and a transfer gate electrode 33, and the potential of the contact electrode 32 is thereafter set to 0 V. Thus, electrons introduced into a transfer channel 14 from the contact electrode 32 through a diffusion layer 16a are stored in an electron storage portion 34b formed under the storage gate electrode 34. Then, the potential of the transfer gate electrode 33 is set to 0 V, and the potential of the contact electrode 32 is set to about 3 V again.

(Period T2)

The node 70 connected to the second electrode 68c is brought into an open state, so that the second electrode 68c is at the same potential as the control gate electrode 35. At least either the first electrode 68b or the second electrode 68c receives infrared radiation and the ferroelectric film 68a accumulates heat, whereby spontaneous polarization of the ferroelectric film 68a is reduced. Thus, a potential difference responsive to the polarization state of the ferroelectric film 68a is caused between the first electrode 68b and the second electrode 68c, to increase the potential of the control gate electrode 35 (second electrode 68c). Consequently, the portion of the transfer channel 14 located under the control gate electrode 35 is brought into an ON-state, and the electrons having been stored in the electron storage portion 34b are transferred to an electron multiplying portion 14a. A transfer operation between the electron multiplying portion 14a and an electron storage portion 14b is performed a plurality of times (about 400 times, for example) similarly to the operations shown in FIGS. 5 and 6, so that the electrons transferred to the electron multiplying portion 14a are multiplied to about 2000 times. Thereafter signal charges resulting from the multiplied electrons are read as a voltage signal through an FD region 18 by the aforementioned read operation.

The effect of the eighth embodiment is similar to that of the aforementioned second embodiment.

### Ninth Embodiment

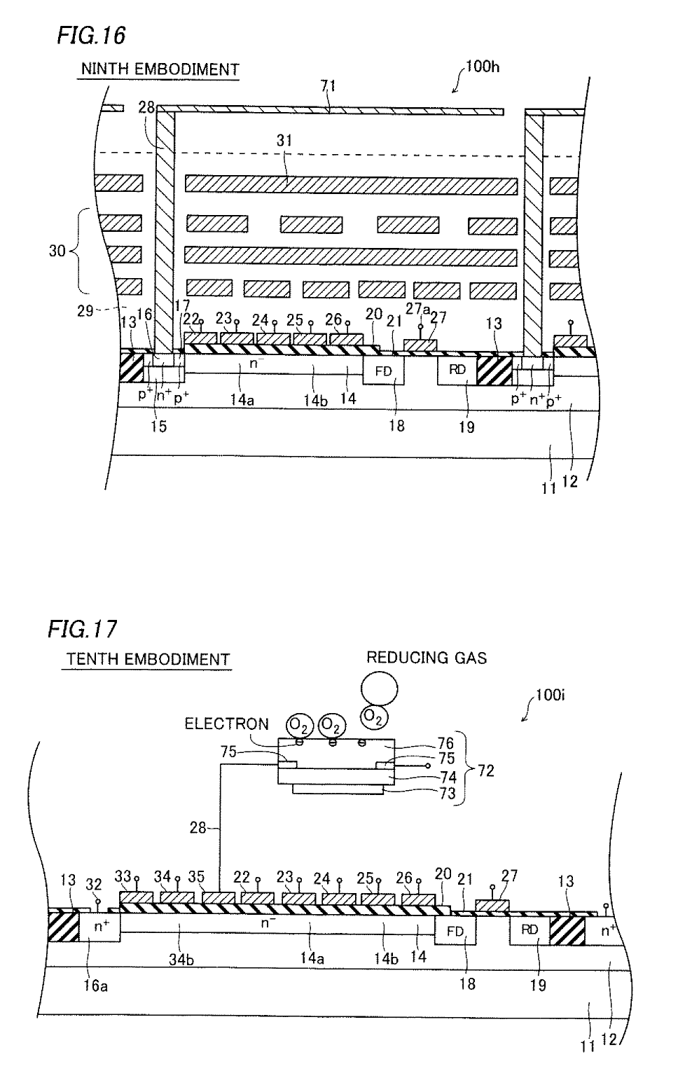

A glucose sensor 100h according to a ninth embodiment of the present invention is now described with reference to FIG. 16. According to the ninth embodiment, the glucose sensor 100h detects glucose, dissimilarly to the aforementioned first to eighth embodiments each formed to detect heat (infrared radiation).

In the glucose sensor 100h according to the ninth embodiment of the present invention, a connecting wire 28 is provided with an electrode 71 made of platinum (Pt), as shown in FIG. 16. The electrode 71 is an example of the “charge supplying portion” in the present invention. According to the ninth embodiment, the glucose sensor 100h is provided with no lower electrode, dissimilarly to the aforementioned first embodiment. The remaining structure of the ninth embodiment is similar to that of the aforementioned first embodiment.

An operation of the glucose sensor 100h detecting glucose is now described.

When glucose and oxygen (O₂) are reacted with each other by glucose oxidase, gluconolactone and hydrogen peroxide (H₂O₂) are formed as follows:

Glucose+O₂→gluconolactone+H₂O₂ (1)

Hydrogen peroxide (H₂O₂) is reacted with the electrode 71 of platinum (Pt), whereby electrons are formed as follows:

H₂O₂→2H⁺+O₂+2e⁻ (2)

The electrons formed by reacting hydrogen peroxide (H₂O₂) with the electrode 71 are supplied to an electron multiplying portion 14a through the connecting wire 28, a diffusion layer 16 and a transfer channel 14, and multiplied.

The glucose sensor 100h according to the ninth embodiment can attain the following effect:

(14) The electrons formed by reacting hydrogen peroxide (H₂O₂) with the electrode 71 containing platinum (Pt) are multiplied by the electron multiplying portion 14a. Thus, sensitivity of the glucose sensor 100b detecting glucose can be easily improved.

### Tenth Embodiment

A semiconductor gas sensor 100i according to a tenth embodiment of the present invention is now described with reference to FIG. 17. According to the tenth embodiment, the semiconductor gas sensor 100i detects reducing gas, dissimilarly to the aforementioned first to eighth embodiments each formed to detect heat (infrared radiation).

In the semiconductor gas sensor 100i according to the tenth embodiment of the present invention, a semiconductor gas sensor portion 72 is connected to a control gate electrode 35 through a connecting wire 28, as shown in FIG. 17. The semiconductor gas sensor portion 72 is constituted of a heater 73 and an alumina substrate 74 provided on the surface of the heater 73 as well as an electrode 75 and an oxide semiconductor (metal oxide) member 76 provided on the surface of the alumina substrate 74. The oxide semiconductor member 76 is made of SnO₂, WO₃, In₂O₃, Fe₂O₃, TiO₂ or the like. The semiconductor gas sensor portion 72 is an example of the “charge supplying portion” in the present invention. The remaining structure of the tenth embodiment is similar to that of the aforementioned second embodiment.

An operation of the semiconductor gas sensor 100i detecting reducing gas is now described.

First, the heater 73 is heated. Thus, oxygen (O₂) is adsorbed to the surface of the oxide semiconductor member 76. Thus, free electrons in the oxide semiconductor member 76 are trapped by oxygen, whereby resistance of the oxide semiconductor member 76 is increased.

When the surface of the oxide semiconductor member 76 comes into contact with reducing gas, oxygen having been adsorbed to the surface of the oxide semiconductor member 76 is reacted with the reducing gas and removed. When the oxide semiconductor member 76 is made of SnO₂, detectable reducing gas is H₂, CO₂, NO₂, H₂S, CH₄ or the like. When the oxide semiconductor member 76 is made of In₂O₃, detectable reducing gas is O₃, NO₂, trimethylamine or the like. When the oxide semiconductor member 76 is made of Fe₂O₃, detectable reducing gas is CO₂, moisture (water vapor) or the like. When the oxide semiconductor member 76 is made of TiO₂, detectable reducing gas is H₂, C₂H₅OH, O₂ or the like.

Oxygen having been adsorbed to the surface of the oxide semiconductor member 76 is reacted with the reducing gas and removed, whereby the resistance of the oxide semiconductor member 76 is reduced. Consequently, a voltage applied from the oxide semiconductor member 76 to the control gate electrode 35 is increased. Electrons corresponding to the increase in the voltage applied to the control gate electrode 35 are stored in an electron storage portion 34b, and the amount of electrons transferred to an electron multiplying portion 14a is increased. The electron multiplying portion 14a multiplies the electrons, thereby detecting the reducing gas.

The semiconductor gas sensor 100i according to the tenth embodiment can attain the following effect:

(15) The electron multiplying portion 14a multiplies electrons corresponding to a current, flowing in the oxide semiconductor member 76, changed due to the reducing gas coming into contact with the surface of the oxide semiconductor member 76. Thus, sensitivity of the semiconductor gas sensor 100i detecting reducing gas can be easily improved.

Although the present invention has been described and illustrated in detail, it is clearly understood that the same is by way of illustration and example only and is not to be taken by way of limitation, the spirit and scope of the present invention being limited only by the terms of the appended claims.

For example, while the sensor according to each of the aforementioned embodiments measures heat (infrared radiation), glucose or reducing gas as the measurement object other than light (particularly visible light), the present invention is not restricted to this. The present invention is also applicable to a sensor detecting an odor, for example. The present invention is further applicable to light such as far-infrared radiation or ultraviolet radiation other than visible light.

While each of the aforementioned embodiments includes the upper and lower electrodes, the pyroelectric detector, the electrode made of platinum (Pt) or the semiconductor gas sensor portion as the example of the charge supplying portion. In each of the aforementioned embodiments, further, electrons are supplied from the charge supplying portion to the diffusion layer, supplied to the control gate electrode as a voltage, or directly supplied to the transfer channel. According to the present invention, the example of the charge supplying portion and the example of the method of supplying electrons are not restricted to the combination of any of the aforementioned embodiments, but may alternatively be in a combination other than the above.

While the transfer gate electrode 22, the multiplier gate electrode 23, the transfer gate electrode 24 and the storage gate electrode 25 are successively arranged on the surface of the transfer channel 14 in this order in each of the aforementioned embodiments, the present invention is not restricted to this. For example, the transfer gate electrode 22, the storage gate electrode 25, the transfer gate electrode 24 and the multiplier gate electrode 23 may alternatively be arranged on the surface of the transfer channel 14 in this order. In other words, the multiplier gate electrode 23 and the storage gate electrode 25 may be interchanged with each other.

While electrons are employed as signal charges in each of the aforementioned embodiments, the present invention is not restricted to this but holes may alternatively be employed as signal charges by entirely reversing the conductivity types of substrate impurities and the polarity of the applied voltage.

While the transfer gate electrode 33 and the control gate electrode 35 are provided in this order to be adjacent to the n⁺-type diffusion layer 16a in the aforementioned third embodiment, the present invention is not restricted to this. According to the present invention, the control gate electrode 35 may be provided to be adjacent to the n⁺-type diffusion layer 16a without providing the transfer gate electrode 33.

What is claimed is:

1. A charge increaser comprising:
a charge supplying portion having a signal source formed by a measurement object other than visible light and supplying signal charges corresponding to said signal source; and
a charge increasing portion for increasing the amount of charges corresponding to said signal charges stored in said charge supplying portion by measuring said measurement object other than visible light.

2. The charge increaser according to claim 1, further comprising a semiconductor substrate provided with said charge increasing portion, wherein
said charge supplying portion is arranged on a portion upwardly or downwardly separated from said semiconductor substrate.

3. The charge increaser according to claim 2, wherein
said charge increasing portion is provided on the upper surface side of said semiconductor substrate, and
said charge supplying portion is provided above said semiconductor substrate, so that said signal charges stored in said charge supplying portion provided above said semiconductor substrate are transferred to said charge increasing portion provided on the upper surface side of said semiconductor substrate and the amount thereof is increased in said charge increasing portion.

4. The charge increaser according to claim 2, wherein
said charge increasing portion is provided on the upper surface side of said semiconductor substrate, and
said charge supplying portion is provided on a portion located under said semiconductor substrate oppositely to the side provided with said charge increasing portion, so that said signal charges stored in said charge supplying portion provided on said portion located under said semiconductor substrate are transferred to said charge increasing portion provided on the upper surface side of said semiconductor substrate and the amount thereof is increased in said charge increasing portion.

5. The charge increaser according to claim 2, further comprising a connecting wire connecting said charge supplying portion and said semiconductor substrate with each other.

6. The charge increaser according to claim 1, wherein
said charge supplying portion and said charge increasing portion are arranged to overlap with each other in plan view.

7. The charge increaser according to claim 6, further comprising a shielding layer provided between said charge supplying portion and said charge increasing portion for suppressing incidence of visible light upon said charge increasing portion.

8. The charge increaser according to claim 1, further comprising:
a charge transfer region functioning as a channel, and
an increasing electrode provided on the surface of said charge transfer region, wherein
said charge increasing portion is provided on said charge transfer region under said increasing electrode, and formed to increase the amount of charges corresponding to said signal charges stored in said charge supplying portion by applying a voltage to said increasing electrode.

9. The charge increaser according to claim 1, wherein
said charge supplying portion includes a first electrode consisting of a plurality of members having different thermal expansion coefficients and a second electrode arranged to be opposed to said first electrode, and
said charge increasing portion is so formed as to increase the amount of charges corresponding to said signal charges resulting from a change in the capacitance between said first electrode and said second electrode caused by thermal deformation of said first electrode.

10. The charge increaser according to claim 1, wherein
said charge supplying portion includes a pyroelectric detector detecting heat and outputting signal charges changed in response to said detected heat, and
said charge increasing portion is so formed as to increase the amount of charges corresponding to said signal charges output from said pyroelectric detector detecting said heat.

11. The charge increaser according to claim 10, wherein
said pyroelectric detector detects heat by infrared radiation and outputs signal charges responsive to the amount of said infrared radiation, so that said charge increasing portion increases the amount of charges corresponding to said signal charges output from said pyroelectric detector.

12. The charge increaser according to claim 1, further comprising:
a semiconductor substrate provided with said charge increasing portion,
a connecting wire connecting said charge supplying portion and said semiconductor substrate with each other, and
an impurity region provided between said connecting wire and said charge transfer region, wherein
said signal charges stored in said charge supplying portion by measuring said measurement object other than visible light are transferred to said charge increasing portion of said charge transfer region through said connecting wire and said impurity region and the amount thereof is increased in said charge increasing portion.

13. The charge increaser according to claim 12, wherein
said impurity region and said connecting wire are directly connected with each other, so that said signal charges stored in said charge supplying portion by measuring said measurement object other than visible light are transferred to said charge increasing portion of said charge transfer region through said connecting wire and said impurity region and the amount thereof is increased in said charge increasing portion.

14. The charge increaser according to claim 1, further comprising:
an impurity region provided to be adjacent to said charge transfer region, and
a transfer electrode provided on the surface of a portion of said charge transfer region located between said impurity region and said increasing electrode in plan view, wherein
a voltage resulting from said signal charges stored in said charge supplying portion by measuring said measurement object other than visible light is applied to said transfer electrode and charges corresponding to said signal charges stored in said charge supplying portion are supplied from said impurity region to said charge transfer region and the amount thereof is increased in said charge increasing portion.

15. The charge increaser according to claim 1, wherein
said signal charges stored in said charge supplying portion by measuring said measurement object other than visible light are directly supplied to said charge transfer region.

16. The charge increaser according to claim 15, wherein
said charge supplying portion includes a pyroelectric detector directly provided on the surface of said charge transfer region for detecting heat and outputting signal charges responsive to said detected heat, and
said signal charges output from said pyroelectric detector detecting said heat are directly supplied from said pyroelectric detector to said charge transfer region and the amount thereof is increased in said charge increasing portion.

17. The charge increaser according to claim 15, further comprising a connecting wire provided between said charge supplying portion and said charge transfer region, wherein
said signal charges stored in said charge supplying portion by measuring said measurement object other than visible light are directly supplied from said connecting wire to said charge transfer region by tunneling and the amount thereof is increased in said charge increasing portion.

18. The charge increaser according to claim 15, further comprising:
a connecting wire provided between said charge supplying portion and said charge transfer region, and
a barrier layer provided between said connecting wire and said charge transfer region for reducing resistance between said connecting wire and said charge transfer region, wherein
said signal charges stored in said charge supplying portion by measuring said measurement object other than visible light are directly supplied to said charge transfer region through said connecting wire and said barrier layer and the amount thereof is increased in said charge increasing portion.

19. The charge increaser according to claim 1, wherein
said charge supplying portion contains platinum, and
said charge increasing portion is so formed as to increase the amount of charges corresponding to signal charges formed by reacting hydrogen peroxide with said charge supplying portion containing platinum.

20. The charge increaser according to claim 1, wherein
said charge supplying portion includes a semiconductor gas sensor containing an oxide semiconductor, and
said charge increasing portion is so formed as to increase the amount of charges corresponding to a current, flowing in said oxide semiconductor, changed by reducing gas coming into contact with the surface of said oxide semiconductor.
